(19) United States
(12) Patent Application Publication
(10) Pub. No.: US 2025/0282698 A1
(43) Pub. Date: Sep. 11, 2025

(54) ANTIFOULANT COMPOSITIONS FOR HIGH-SEVERITY PROCESSING OF VINYLIC MONOMER STREAMS

(71) Applicant: ECOLAB USA Inc., St. Paul, MN (US)

(72) Inventors: Jonathan MASERE, Richmond, TX (US); Zhenxing XI, Katy, TX (US); Pedro Carvalho CAMPOS, Perre (PT); David DIXON, Baton Rouge, LA (US); Gregory Appel MELANCON, JR., Baton Rouge, LA (US); Ramon COLORADO, JR., Stafford, TX (US)

(73) Assignee: ECOLAB USA Inc., St. Paul, MN (US)

(21) Appl. No.: 19/217,605

(22) Filed: May 23, 2025

Related U.S. Application Data
(63) Continuation of application No. 18/191,225, filed on Mar. 28, 2023, now Pat. No. 12,344,581.
(60) Provisional application No. 63/326,373, filed on Apr. 1, 2022.

(51) Int. Cl. C07C 7/20; C07C 11/167; C07C 11/18; C07C 13/15; C07C 15/46; C07C 51/50; C07C 57/04; C07C 67/62; C07C 69/15; C07C 211/51; C07D 221/00

(52) U.S. Cl. CPC C07C 7/20; C07C 51/50; C07C 67/62; C07C 211/51; C07D 221/00; C07C 11/167; C07C 11/18; C07C 13/15; C07C 15/46; C07C 57/04; C07C 69/15

(57) ABSTRACT

Inhibitor compositions for abating undesirable polymerization during processing of hydrocarbon stream laden with reactive vinylic monomers are provided. The polymerization inhibitor compositions include at least a first inhibitor compound having a stable nitroxide radical and a second inhibitor including phenylenediamine. Methods of inhibiting the polymerization of monomers using the compositions of the disclosure are also provided. The methods of inhibiting polymerization of monomers include a step of adding a composition of the disclosure to the monomer. In some instances, the monomer is an ethylenically unsaturated monomer. Such ethylenically unsaturated monomers include, but are not limited to, vinyl acetate, acrylonitrile, acrylates, methacrylates, 1,3-butadiene, styrene, isoprene, (meth)acrylic acid, and combinations thereof. Methods of preparing the polymerization inhibitors and compositions of the disclosure are also provided.

### FIELD OF THE INVENTION

The present disclosure generally relates to compositions that include a blend of polymerization inhibitors and methods of using the same. More particularly, the present disclosure relates to compositions that include at least one compound having a stable nitroxide radical and phenylenediamine, useful for inhibiting polymerization of ethylenic unsaturated monomers.

### BACKGROUND

The manufacture of ethylenically unsaturated monomers typically comprises three stages: reaction, crude product recovery, and product purification through fractional distillation. Distillation operations, performed at elevated temperatures, are often involved in the recovery and the purification stages. Ethylenically unsaturated monomers, such as styrene, butadiene, isoprene, divinyl benzene, cyclopentadiene, dicyclopentadiene, vinyl acetate, acrylate, and methacrylate monomers, are present in crude process streams or in refined products made by various chemical industrial processes. However, said monomers have high reactivities, especially at elevated temperatures, in the presence of oxygen or when in contact with metal oxide surfaces. Thus, these monomer types are highly prone to undesirably polymerize through radical polymerization. This problem is acute, especially at elevated temperatures and in the presence of polymerization initiators such as organic peroxides. The resulting polymers can be problematic and lead to equipment “fouling” and product contamination and consumption. Production efficiency decreases once the resultant polymer precipitates out of solution during the processing stage and is deposited onto equipment surfaces. As a consequence of the fouling of the process equipment, operations have to be halted to mechanically clean the equipment and/or to remove the undesired polymers. Operational stoppages lead to substantial financial losses for the operators. The polymer can also remain in solution as a soluble product contaminant. Contamination may necessitate additional processing steps to remove the contaminant polymer from the final product compositions streams or stored product. All of the above-mentioned problems have rendered it imperative to develop and use online chemical cleaning procedures to mitigate fouling and thereby eliminating the financially costly shutting down of operations.

The premature polymerization of these monomers is generally abated by dosing polymerization inhibitors capable of eliminating or significantly reducing the premature polymerization of the monomers. Conventional polymerization inhibitors include stable free radicals that can effectively scavenge carbon-centered radicals. As operators are now using feedstocks that demand high-severity operational conditions, the use of conventional inhibitors is proving to be inefficacious. Conventional polymerization inhibitors, such as 4-hydroxy-2,2,6,6-tetramethylpiperidin-1-oxyl (HTEMPO) and 4-oxo-2,2,6,6-tetramethylpiperidin-1-oxyl (OTEMPO), generally degrade and lose their efficacy as polymerization inhibitors under high-temperature processing environments. Consequently, there is a pressing demand for the development of new polymerization inhibitor compositions that have high thermal stability and, therefore, not liable to loss of inhibitory functions at elevated process temperatures.

### BRIEF SUMMARY

A composition for inhibiting monomer polymerization is provided. The composition includes a first inhibitor compound comprising a stable nitroxide radical; and a second inhibitor compound comprising a phenylenediamine.

In some aspects, the first inhibitor compound is of formula (I):

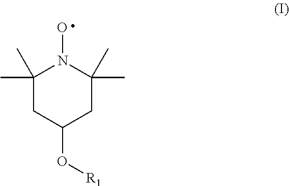

wherein R₁ is C₁-C₂₂ alkyl or aryl, wherein the alkyl and aryl are optionally substituted with one or more C₁-C₂₂ alkyl or aryl.

In some aspects, the first inhibitor is selected from the group consisting of: 1-oxyl-2,2,6,6-tetramethylpiperin-4-ol; 4-methoxy-2,2,6,6-tetramethylpiperidin-1-oxy; 4-ethoxy-2,2,6,6-tetramethylpiperidin-1-oxy; 4-propoxy-2,2,6,6-tetramethylpiperidin-1-oxy; 4-butoxy-2,2,6,6-tetramethylpiperidin-1-oxy; 4-pentoxy-2,2,6,6-tetramethylpiperidin-1-oxy; 4-hexyloxy-2,2,6,6-tetramethylpiperidin-1-oxy; 4-heptyloxy-2,2,6,6-tetramethylpiperidin-1-oxy; 4-octyloxy-2,2,6,6-tetramethylpiperidin-1-oxy; 4-nonyloxy-2,2,6,6-tetramethylpiperidin-1-oxy; 4-decyloxy-2,2,6,6-tetramethylpiperidin-1-oxy; 4-undecyloxy-2,2,6,6-tetramethylpiperidin-1-oxy; 4-dodecyloxy-2,2,6,6-tetramethylpiperidin-1-oxy; 4-tridecyloxy-2,2,6,6-tetramethylpiperidin-1-oxy; 4-tetradecyloxy-2,2,6,6-tetramethylpiperidin-1-oxy; 4-pentadecyloxy-2,2,6,6-tetramethylpiperidin-1-oxy; 4-hexadecyloxy-2,2,6,6-tetramethylpiperidin-1-oxy; 4-heptadecyloxy-2,2,6,6-tetramethylpiperidin-1-oxy; 4-octadecyloxy-2,2,6,6-tetramethylpiperidin-1-oxy; 4-nodecyloxy-2,2,6,6-tetramethylpiperidin-1-oxy; 4-decyloxy-2,2,6,6-tetramethylpiperidin-1-oxy; 4-icosyloxy-2,2,6,6-tetramethylpiperidin-1-oxy; 4-henicosyloxy-2,2,6,6-tetramethylpiperidin-1-oxy; 4-docosyloxy-2,2,6,6-tetramethylpiperidin-1-oxy; 4-(phenoxy)2,2,6,6-tetramethylpiperidin-1-oxy; 4-(benzyloxy)-2,2,6,6-tetramethylpiperidin-1-oxy; 2,2,6,6-tetramethyl-4-(naphthalen-2-yloxy)piperidin-1-oxy; and any combination thereof.

In some aspects, the first inhibitor is a compound of formula III:

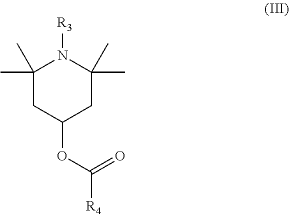

wherein R₃ is —O· or —OH; and R₄ is C₁-C₂₂ alkyl or aryl, wherein the alkyl and aryl are optionally substituted with one or more C₁-C₂₂ alkyl or aryl.

In some aspects, the first inhibitor is selected from the group consisting of: 1-oxyl-2,2,6,6-tetramethylpiperidin-4-yl acetate; 1-oxyl-2,2,6,6-tetramethylpiperidin-4-yl propanoate; 1-oxyl-2,2,6,6-tetramethylpiperidin-4-yl butyrate; 1-oxyl-2,2,6,6-tetramethylpiperidin-4-yl pentanoate; 1-oxyl-2,2,6,6-tetramethylpiperidin-4-yl hexanoate; 1-oxyl-2,2,6,6-tetramethylpiperidin-4-yl heptanoate; 1-oxyl-2,2,6,6-tetramethylpiperidin-4-yl octanoate; 1-oxyl-2,2,6,6-tetramethylpiperidin-4-yl nonanoate; 1-oxyl-2,2,6,6-tetramethylpiperidin-4-yl decanoate; 1-oxyl-2,2,6,6-tetramethylpiperidin-4-yl undecanoate; 1-oxyl-2,2,6,6-tetramethylpiperidin-4-yl dodecanoate; 1-oxyl-2,2,6,6-tetramethylpiperidin-4-yl 2-ethylhexanoate; 1-oxyl-2,2,6,6-tetramethylpiperidin-4-yl stearate; 1-oxyl-2,2,6,6-tetramethylpiperidin-4-yl benzoate; 1-oxyl-2,2,6,6-tetramethylpiperidin-4-yl palmitoate; 1-oxyl-2,2,6,6-tetramethylpiperidin-4-yl behenoate; 1-oxyl-2,2,6,6-tetramethylpiperidin-4-yl 4-tert-butylbenzoate; and any combination thereof.

In some aspects, the second inhibitor compound is a phenylenediamine of formula (IV) or formula (V):

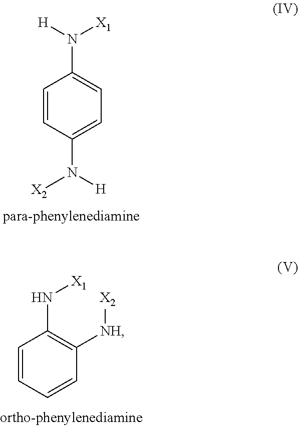

wherein X₁ and X₂ are independently C₁-C₂₂ alkyl or aryl, wherein the alkyl and aryl are optionally substituted with one or more C₁-C₂₂ alkyl or aryl.

In some aspects, X₁ and X₂ are independently C₁-C₂₂ alkyl or phenyl, wherein the alkyl and phenyl are optionally substituted with one or more C₁-C₂₂ alkyl or aryl.

In some aspects, X₁ and X₂ are independently C₁-C₁₀ alkyl or phenyl, wherein the alkyl and phenyl are optionally substituted with one or more C₁-C₂₂ alkyl or aryl.

In some aspects, X₁ and X₂ are independently C₁-C₅ alkyl or phenyl, wherein the alkyl and phenyl are optionally substituted with one or more C₁-C₂₂ alkyl or aryl.

In some aspects, the second inhibitor is selected from the group consisting of: 1,2-phenylenediamine, 1,4-phenylenediamine, N,N′-di-methyl-p-phenylenediamine, N,N′-di-sec-butyl-1,4-phenylenediamine, N,N′-di-1,4-dimethylpentyl-1,4-phenylenediamine, N, N′-di-acetyl-1,4-phenylenediamine, N-tert-butyl-N′-phenyl-1,4-phenylenediamine, N,N′-di-phenyl-1,4-phenylenediamine, and any combination thereof.

In some aspects, the first inhibitor compound is present in the composition at a concentration of about 0.01% by weight to about 80% by weight.

In some aspects, the second inhibitor compound is present in the composition at a concentration of about 0.01% by weight to about 50% by weight.

In some aspects, a mole ratio of the first inhibitor compound to the second inhibitor compound is about 100:1 to about 1:100.

In some aspects, the composition further comprises an organic solvent.

In some aspects, the composition further comprises an ethylenic unsaturated monomer selected from the group consisting of vinyl acetate, acrylonitrile, an acrylate, a methacrylate, 1,3-butadiene, styrene, isoprene, acrylic acid, methacrylic acid and any combination thereof.

A method of inhibiting polymerization of a monomer is provided. The method includes adding any composition described herein to the monomer.

In some aspects, the monomer is provided within a solution.

In some aspects, the solution further comprises one or more additional components selected from: an acid, an organic solvent, and water.

In some aspects, the monomer is an ethylenic unsaturated monomer.

In some aspects, the composition is added to the monomer such that a concentration of the first inhibitor compound is about 0.1 ppm to about 10,000 ppm.

In some aspects, the composition is added to the monomer such that a concentration of the second inhibitor compound is about 0.1 ppm to about 10,000 ppm.

In some aspects, the monomer is selected from the group consisting of vinyl acetate, acrylonitrile, an acrylate, a methacrylate, 1,3-butadiene, styrene, divinyl benzene, isoprene, cyclopentadiene, dicyclopentadiene, acrylic acid, methacrylic acid and any combination thereof.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific aspects disclosed may be readily utilized as a basis for modifying or designing other aspects for carrying out the same purposes of the present disclosure. It should also be realized by those skilled in the art that such equivalent aspects do not depart from the spirit and scope of the disclosure as set forth in the appended claims.

### BRIEF DESCRIPTION OF THE DRAWINGS

A detailed description of the invention is hereafter described with specific reference being made to the drawings.

FIG. 1 shows The comparative polymerization kinetics of styrene solutions, untreated and treated, under high severity reaction temperature of 135° C. under static test conditions.

FIG. 2 shows amount of polystyrene formed after 6 hour of polymerization reaction at a high severity temperature of about 135° C.

FIG. 3 shows comparative styrene polymerization reaction kinetics under high severity conditions.

### DETAILED DESCRIPTION

Various aspects of the present disclosure are described below. The relationship and functioning of the various elements of the aspects may better be understood by reference to the following detailed description. However, aspects are not limited to those explicitly described herein and it should be understood that, in certain instances, details may have been omitted that are not necessary for an understanding of the aspects disclosed herein, such as—for example—conventional synthesis and/or formulation.

By increasing the process temperature above the typical range, antifoulants that had previously proven to be effective have failed. New operational conditions are characterized as high severity. Prior to the increase in the temperature into the high-severity regime, operations were running for more than 8 years without emergency shut downs to remove foulant material from process equipment. As soon as the operating temperature was increased above the typical upper-limit threshold, the rate of fouling increased drastically. Too frequently, operations have had to be stopped as a matter of emergency. Such unscheduled operational stoppages are costly. To prevent this, the development of high-temperature inhibitors was necessary.

As used herein “high severity” refers to conditions of a distillation process where the heating medium has a temperature above a certain value. Depending on the heating medium the temperature at which “high severity” conditions begin can be different. When the heating medium is water/condensate, a temperature of at least about 100° C. can be considered “high severity.” When the heating medium is low pressure steam, a temperature of at least about 125° C. can be considered “high severity.” When the heating medium is intermediate pressure steam, a temperature of at least about 160° C. can be considered “high severity.” When the heating medium is high pressure steam, a temperature of at least about 180° C. can be considered “high severity.” When the heating medium is oil, a temperature of at least about 300° C. can be considered “high severity.”

The present disclosure relates to compositions that include a blend of polymerization inhibitors and methods of using the same to inhibit the polymerization of ethylenic unsaturated monomers. Polymerization inhibitor compositions of the present disclosure include at least one compound having a thermally and chemically stable nitroxide radical and a phenylenediamine. The polymerization inhibitor compositions can be blends of multiple components, including components in addition to the aforementioned compounds having a stable nitroxide radical and a phenylenediamine.

A “polymerization inhibitor,” in the presence of polymerizable monomers, inhibits the polymerization of these monomers during the induction time under shutdown conditions. After the induction time has elapsed following the complete consumption of the polymer inhibitor, the polymer's formation occurs at the same rate as is the case in the total absence of the polymerization inhibitor.

Polymerization inhibitors and polymerization retarders can be considered generally as “antipolymerants” which are compounds that can inhibit or reduce the formation of polymers from one or more radically polymerizable compounds.

The term “fouling” refers to the formation of polymers, prepolymers, oligomer and/or other materials, which would become insoluble in and/or precipitate from a stream and deposit on equipment under the conditions of operation of the equipment. In turn, the inhibitor compositions of the disclosure can be referred to as “antifouling” as they inhibit or reduce the formation of foulant polymers.

### Compositions of the Disclosure

The present disclosure relates to compositions for inhibiting monomer polymerization where the compositions include a first inhibitor compound having a stable nitroxide radical and a second inhibitor compound a phenylenediamine. In some aspects, the compositions used herein are especially useful in high severity conditions in distillation columns. In some aspects, the compositions are for inhibiting monomer polymerization, where the monomer is an ethylenic unsaturated monomer. For example, the compositions of the disclosure are useful for inhibiting polymerization of ethylenic unsaturated monomers including, but not limited to, vinyl acetate, acrylonitrile, acrylate esters, methacrylate esters, 1,3-butadiene, styrene, isoprene, acrylic acid, (meth)acrylic acid, and combinations thereof.

In some aspects, the compositions of the disclosure are useful for inhibiting the polymerization of ethylenic unsaturated monomers at high-severity operational conditions.

In some aspects, the first inhibitor compound having a stable nitroxide radical is a compound of formula (I):

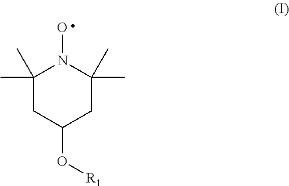

where R₁ is H, C₁-C₂₂ alkyl, C₁-C₂₂ alkenyl, C₁-C₂₂ alkynyl, C₁-C₂₂ cycloalkyl, aryl, —C₁-C₂₂ alkylene aryl, —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), or —C(O)(C₁-C₂₂ alkylene aryl), where the alkyl, cycloalkyl, and aryl are optionally substituted with one or more C₁-C₂₂ alkyl, C₁-C₂₂ alkenyl, C₁-C₂₂ alkynyl, or aryl.

The term “aryl” refers to monocyclic, bicyclic (fused), and tricyclic (fused or spiro) hydrocarbon ring systems having a total of five to fourteen ring carbon atoms, wherein at least one ring in the system is aromatic and wherein each ring in the system contains 3 to 7 ring carbon atoms. The term “aryl” may be used interchangeably with the term “aryl ring”.

In certain aspects, R₁ is —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), or —C(O)(C₁-C₂₂ alkylene aryl), wherein the alkyl, cycloalkyl, and aryl are optionally substituted with one or more C₁-C₂₂ alkyl, C₁-C₂₂ alkenyl, C₁-C₂₂ alkynyl, and aryl.

In some aspects, R₁ is C₁-C₂₂ alkyl or aryl, wherein the alkyl and aryl are optionally substituted with one or more C₁-C₂₂ alkyl or aryl.

In some aspects, R₁ is H. In some aspects, R₁ is C₁-C₂₂ alkyl. In some aspects, R₁ is C₁-C₂₂ alkenyl. In some aspects, R₁ is C₁-C₂₂ alkynyl. In some aspects, R₁ is C₁-C₂₂ cycloalkyl, where the cycloalkyl is optionally substituted with one or more C₁-C₂₂ alkyl, C₁-C₂₂ alkenyl, C₁-C₂₂ alkynyl, or aryl. In some aspects, R₁ is aryl, where the aryl is optionally substituted with one or more C₁-C₂₂ alkyl, C₁-C₂₂ alkenyl, C₁-C₂₂ alkynyl, or aryl. In some aspects, R₁ is —C₁-C₂₂ alkylene aryl, where the aryl is optionally substituted with one or more C₁-C₂₂ alkyl, C₁-C₂₂ alkenyl, C₁-C₂₂ alkynyl, or aryl. In some aspects, R₁ is —C(O)(C₁-C₂₂ alkyl). In some aspects, R₁ is —C(O)(C₁-C₁₂ alkyl). In some aspects, R₁ is —C(O)(C₁-C₆ alkyl). In some aspects, R₁ is —C(O)(methyl). In some aspects, R₁ is —C(O)(ethyl). In some aspects, R₁ is —C(O)(propyl). In some aspects, R₁ is —C(O)(butyl). In some aspects, R₁ is —C(O)(C₁-C₂₂ alkenyl). In some aspects, R₁ is —C(O)(C₁-C₂₂ alkynyl). In some aspects, R₁ is —C(O)(C₁-C₂₂ cycloalkyl), where the cycloalkyl is optionally substituted with one or more C₁-C₂₂ alkyl, C₁-C₂₂ alkenyl, C₁-C₂₂ alkynyl, or aryl. In some aspects, R₁ is —C(O)(aryl), where the aryl is optionally substituted with one or more C₁-C₂₂ alkyl, C₁-C₂₂ alkenyl, C₁-C₂₂ alkynyl, or aryl. In some aspects, R₁ is —C(O)(C₁-C₂₂ alkylene aryl), where the aryl is optionally substituted with one or more C₁-C₂₂ alkyl, C₁-C₂₂ alkenyl, C₁-C₂₂ alkynyl, or aryl.

Examples of compounds of formula (I) include, but are not limited to, 1-oxyl-2,2,6,6-tetramethylpiperin-4-ol; 4-methoxy-2,2,6,6-tetramethylpiperidin-1-oxy; 4-ethoxy-2,2,6,6-tetramethylpiperidin-1-oxy; 4-propoxy-2,2,6,6-tetramethylpiperidin-1-oxy; 4-butoxy-2,2,6,6-tetramethylpiperidin-1-oxy; 4-pentoxy-2,2,6,6-tetramethylpiperidin-1-oxy; 4-hexyloxy-2,2,6,6-tetramethylpiperidin-1-oxy; 4-heptyloxy-2,2,6,6-tetramethylpiperidin-1-oxy; 4-octyloxy-2,2,6,6-tetramethylpiperidin-1-oxy; 4-nonyloxy-2,2,6,6-tetramethylpiperidin-1-oxy; 4-decyloxy-2,2,6,6-tetramethylpiperidin-1-oxy; 4-undecyloxy-2,2,6,6-tetramethylpiperidin-1-oxy; 4-dodecyloxy-2,2,6,6-tetramethylpiperidin-1-oxy; 4-tridecyloxy-2,2,6,6-tetramethylpiperidin-1-oxy; 4-tetradecyloxy-2,2,6,6-tetramethylpiperidin-1-oxy; 4-pentadecyloxy-2,2,6,6-tetramethylpiperidin-1-oxy; 4-hexadecyloxy-2,2,6,6-tetramethylpiperidin-1-oxy; 4-heptadecyloxy-2,2,6,6-tetramethylpiperidin-1-oxy; 4-octadecyloxy-2,2,6,6-tetramethylpiperidin-1-oxy; 4-nodecyloxy-2,2,6,6-tetramethylpiperidin-1-oxy; 4-decyloxy-2,2,6,6-tetramethylpiperidin-1-oxy; 4-icosyloxy-2,2,6,6-tetramethylpiperidin-1-oxy; 4-henicosyloxy-2,2,6,6-tetramethylpiperidin-1-oxy; 4-docosyloxy-2,2,6,6-tetramethylpiperidin-1-oxy; 4-(phenoxy)2,2,6,6-tetramethylpiperidin-1-oxy; 4-(benzyloxy)-2,2,6,6-tetramethylpiperidin-1-oxy; or 2,2,6,6-tetramethyl-4-(naphthalen-2-yloxy)piperidin-1-oxy.

In other aspects, the first inhibitor compound is of formula (II):

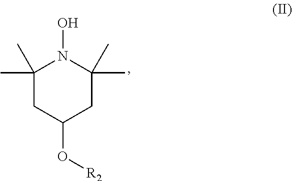

where R₂ is selected from H, C₁-C₂₂ alkyl, C₁-C₂₂ alkenyl, C₁-C₂₂ alkynyl, C₁-C₂₂ cycloalkyl, aryl, —C₁-C₂₂ alkylene, —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), and —C(O)(C₁-C₂₂ alkylene), wherein the alkyl, alkylene, cycloalkyl, and aryl are optionally substituted with one or more C₁-C₂₂ alkyl, C₁-C₂₂ alkenyl, C₁-C₂₂ alkynyl, or aryl.

In certain aspects, R₂ is —C(O)(C₁-C₂₂ alkyl), —C(O)(C₁-C₂₂ alkenyl), —C(O)(C₁-C₂₂ alkynyl), —C(O)(C₁-C₂₂ cycloalkyl), —C(O)(aryl), and —C(O)(C₁-C₂₂ alkylene), where the alkyl, alkylene, cycloalkyl, and aryl are optionally substituted with one or more C₁-C₂₂ alkyl, C₁-C₂₂ alkenyl, C₁-C₂₂ alkynyl, or aryl.

In some aspects, R₂ is H. In some aspects, R₂ is C₁-C₂₂ alkyl. In some aspects, R₂ is C₁-C₂₂ alkenyl. In some aspects, R₂ is C₁-C₂₂ alkynyl. In some aspects, R₂ is C₁-C₂₂ cycloalkyl, where the cycloalkyl is optionally substituted with one or more C₁-C₂₂ alkyl, C₁-C₂₂ alkenyl, C₁-C₂₂ alkynyl, or aryl. In some aspects, R₂ is aryl, where the aryl is optionally substituted with one or more C₁-C₂₂ alkyl, C₁-C₂₂ alkenyl, C₁-C₂₂ alkynyl, or aryl. In some aspects, R₂ is —C₁-C₂₂ alkylene, where the alkylene is optionally substituted with aryl that is optionally substituted with one or more C₁-C₂₂ alkyl, C₁-C₂₂ alkenyl, C₁-C₂₂ alkynyl, or aryl. In some aspects, R₂ is —C(O)(C₁-C₂₂ alkyl). In some aspects, R₂ is —C(O)(C₁-C₁₂ alkyl). In some aspects, R₂ is —C(O)(C₁-C₆ alkyl). In some aspects, R₂ is —C(O)(methyl). In some aspects, R₂ is —C(O)(ethyl). In some aspects, R₂ is —C(O)(propyl). In some aspects, R₂ is —C(O)(butyl). In some aspects, R₂ is —C(O)(C₁-C₂₂ alkenyl). In some aspects, R₂ is —C(O)(C₁-C₂₂ alkynyl). In some aspects, R₂ is —C(O)(C₁-C₂₂ cycloalkyl), where the cycloalkyl is optionally substituted with one or more C₁-C₂₂ alkyl, C₁-C₂₂ alkenyl, C₁-C₂₂ alkynyl, or aryl. In some aspects, R₂ is —C(O)(aryl), where the aryl is optionally substituted with one or more C₁-C₂₂ alkyl, C₁-C₂₂ alkenyl, C₁-C₂₂ alkynyl, or aryl. In some aspects, R₂ is —C(O)(C₁-C₂₂ alkylene), where the alkylene is optionally substituted with aryl that is optionally substituted with one or more C₁-C₂₂ alkyl, C₁-C₂₂ alkenyl, C₁-C₂₂ alkynyl, or aryl.

In some aspects, the compound of formula (II) is 2,2,6,6-tetramethylpiperin-1,4-diol; 4-methoxy-2,2,6,6-tetramethylpiperidin-1-ol; 4-ethoxy-2,2,6,6-tetramethylpiperidin-1-ol; 4-propoxy-2,2,6,6-tetramethylpiperidin-1-ol; 4-butoxy-2,2,6,6-tetramethylpiperidin-1-ol; 4-pentoxy-2,2,6,6-tetramethylpiperidin-1-ol; 4-hexyloxy-2,2,6,6-tetramethylpiperidin-1-ol; 4-heptyloxy-2,2,6,6-tetramethylpiperidin-1-ol; 4-octyloxy-2,2,6,6-tetramethylpiperidin-1-ol; 4-nonyloxy-2,2,6,6-tetramethylpiperidin-1-ol; 4-decyloxy-2,2,6,6-tetramethylpiperidin-1-ol; 4-undecyloxy-2,2,6,6-tetramethylpiperidin-1-ol; 4-dodecyloxy-2,2,6,6-tetramethylpiperidin-1-ol; 4-tridecyloxy-2,2,6,6-tetramethylpiperidin-1-ol; 4-tetradecyloxy-2,2,6,6-tetramethylpiperidin-1-ol; 4-pentadecyloxy-2,2,6,6-tetramethylpiperidin-1-ol; 4-hexadecyloxy-2,2,6,6-tetramethylpiperidin-1-ol; 4-heptadecyloxy-2,2,6,6-tetramethylpiperidin-1-ol; 4-octadecyloxy-2,2,6,6-tetramethylpiperidin-1-ol; 4-nodecyloxy-2,2,6,6-tetramethylpiperidin-1-ol; 4-decyloxy-2,2,6,6-tetramethylpiperidin-1-ol; 4-icosyloxy-2,2,6,6-tetramethylpiperidin-1-ol; 4-henicosyloxy-2,2,6,6-tetramethylpiperidin-1-ol; 4-docosyloxy-2,2,6,6-tetramethylpiperidin-1-ol; 4-(phenoxy)-2,2,6,6-tetramethylpiperidin-1-ol; 4-(benzyloxy)-2,2, 6,6-tetramethylpiperidin-1-ol; or 2,2,6,6-tetramethyl-4-(naphthalen-2-yloxy)piperidin-1-ol.

In certain aspects, the compositions of the disclosure include compounds of formula (I) and (II), respectively, where R₁ and R₂ are the same. For example, in some aspects, the compositions of the disclosure include compounds of formula (I) and (II), respectively, where R₁ and R₂ are each, independently, —C(O)(C₁-C₂₂ alkyl). In certain aspects, the compositions of the disclosure include first and second inhibitor compounds of formula (I) and (II), respectively, where R₁ and R₂ are different.

The presently disclosed compound of formula (II) having a hydroxylamine has benefits over the corresponding nitroxide (compound of formula (I)), such as the capability to provide additional polymerization inhibition, as will be more fully explained below. A general synthetic route to produce a hydroxylamine of a nitroxide is to reduce its corresponding nitroxide with a reducing reagent as follows:

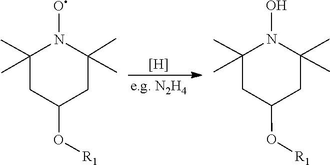

A hydroxylamine of a nitroxide has the potential to provide additional polymerization inhibition as compared to the corresponding nitroxide when carbon-centered and oxygen-centered radical initiators are present. This is explained as follows:

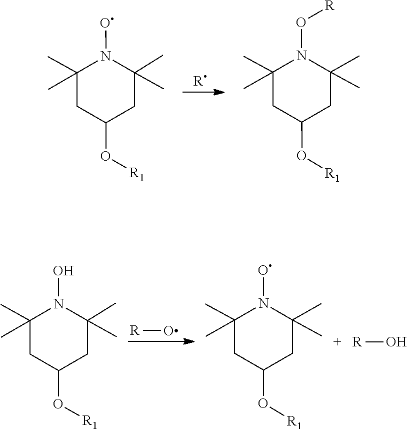

The hydroxylamine of a nitroxide is an excellent hydrogen donor due to its weak NO—H bond in the compound, and thus it is an efficient antioxidant. As an antioxidant, the hydroxylamine of a nitroxide easily reacts with oxygen-centered radicals, such as peroxide radicals, while it's converted to its corresponding nitroxide. Nitroxides are generally known as the most effective inhibitors because of their superior inhibiting capabilities through scavenging carbon-centered free radicals at a nearly diffusion controlled rate. This rate is several orders of magnitude faster than phenolic compounds. However, their kinetic superiority is not always advantageous. For instance, it may lose its superiority when oxygen-centered radicals are present as the predominant free radicals. Another issue of concern with a nitroxide is its consumption through non-inhibition and unwanted reactions with process stream components or other inhibitor additives. As a result, high nitroxide inhibitor dosages are often required for a given inhibition efficacy, thereby making their use economically unattractive or even infeasible.

In essence, each hydroxylamine of a nitroxide is equivalent to one hydrogen donor plus one nitroxide antipolymerant when oxygen-centered radicals and carbon-centered radicals are both present, which is an attractive incentive offered by the hydroxylamines of nitroxides. That is, one hydroxylamine of a nitroxide is able to eliminate one oxygen-centered radical and one carbon-centered radical whereas a nitroxide is only capable to eliminate a carbon-centered radical.

In some aspects, the first inhibitor is a compound of formula III:

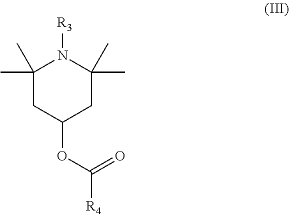

wherein R₃ is —O· or —OH; and R₄ is C₁-C₂₂ alkyl or aryl, wherein the alkyl and aryl are optionally substituted with one or more C₁-C₂₂ alkyl or aryl.

In some aspects, R₃ is —O·. In some aspects, R₃ is —OH.

In some aspects, R₄ is C₁-C₂₂ alkyl that is optionally substituted with one or more C₁-C₂₂ alkyl or aryl. In some aspects, R₄ is aryl that is optionally substituted with one or more C₁-C₂₂ alkyl or aryl.

Examples of a compound of formula (III) include, but are not limited to, 1-oxyl-2,2,6,6-tetramethylpiperidin-4-yl acetate; 1-oxyl-2,2,6,6-tetramethylpiperidin-4-yl propanoate; 1-oxyl-2,2,6,6-tetramethylpiperidin-4-yl butyrate; 1-oxyl-2,2,6,6-tetramethylpiperidin-4-yl pentanoate; 1-oxyl-2,2,6,6-tetramethylpiperidin-4-yl hexanoate; 1-oxyl-2,2,6,6-tetramethylpiperidin-4-yl heptanoate; 1-oxyl-2,2,6,6-tetramethylpiperidin-4-yl octanoate; 1-oxyl-2,2,6,6-tetramethylpiperidin-4-yl nonanoate; 1-oxyl-2,2,6,6-tetramethylpiperidin-4-yl decanoate; 1-oxyl-2,2,6,6-tetramethylpiperidin-4-yl undecanoate; 1-oxyl-2,2,6,6-tetramethylpiperidin-4-yl dodecanoate; 1-oxyl-2,2,6,6-tetramethylpiperidin-4-yl 2-ethylhexanoate; 1-oxyl-2,2,6,6-tetramethylpiperidin-4-yl stearate; 1-oxyl-2,2,6,6-tetramethylpiperidin-4-yl benzoate; 1-oxyl-2,2,6,6-tetramethylpiperidin-4-yl palmitoate; 1-oxyl-2,2,6,6-tetramethylpiperidin-4-yl behenoate; or 1-oxyl-2,2,6,6-tetramethylpiperidin-4-yl 4-tert-butylbenzoate.

In some aspects, the second inhibitor compound is a phenylenediamine of formula (IV) or formula (V):

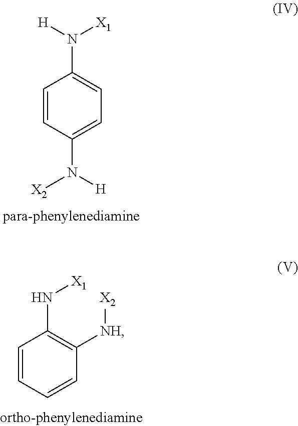

wherein X₁ and X₂ are independently C₁-C₂₂ alkyl, or aryl, wherein the alkyl and aryl are optionally substituted with one or more C₁-C₂₂ alkyl or aryl.

In some aspects, the second inhibitor compound is a phenylenediamine of formula (IV). In some aspects, the second inhibitor compound is a phenylenediamine of formula (V).

Examples of phenylenediamines include, but are not limited to, 1,2-phenylenediamine, 1,4-phenylenediamine, N,N′-di-methyl-p-phenylenediamine, N,N′-di-sec-butyl-1,4-phenylenediamine, N,N′-di-1,4-dimethylpentyl-1,4-phenylenediamine, N, N′-di-acetyl-1,4-phenylenediamine, N-tert-butyl-N′-phenyl-1,4-phenylenediamine, and N,N′-di-phenyl-1,4-phenylenediamine.

In some aspects, the composition consists essentially of a first inhibitor compound and a second inhibitor compound. In other aspects, the composition consists of an organic solvent, a first inhibitor, and a second inhibitor.

In some aspects, the first inhibitor compound is present in the composition at a concentration of about 0.01% by weight to about 80% by weight. In some aspects, the first inhibitor compound is present in the composition at a concentration of about 0.01% by weight to about 70% by weight. In some aspects, the first inhibitor compound is present in the composition at a concentration of about 0.01% by weight to about 60% by weight. In some aspects, the first inhibitor compound is present in the composition at a concentration of about 0.01% by weight to about 50% by weight. In some aspects, the first inhibitor compound is present in the composition at a concentration of about 0.01% by weight to about 40% by weight. In some aspects, the first inhibitor compound is present in the composition at a concentration of about 0.01% by weight to about 30% by weight. In some aspects, the first inhibitor compound is present in the composition at a concentration of about 0.01% by weight to about 20% by weight. In some aspects, the first inhibitor compound is present in the composition at a concentration of about 0.01% by weight to about 10% by weight.

For example, in certain aspects, the first inhibitor compound is present in the composition at a concentration of about 0.01% by weight, about 0.1% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, or about 80% by weight.

In some aspects, the second inhibitor compound is present in the composition at a concentration of about 0.01% by weight to about 50% by weight. In some aspects, the second inhibitor compound is present in the composition at a concentration of about 0.01% by weight to about 40% by weight. In some aspects, the second inhibitor compound is present in the composition at a concentration of about 0.01% by weight to about 30% by weight. In some aspects, the second inhibitor compound is present in the composition at a concentration of about 0.01% by weight to about 20% by weight. In some aspects, the second inhibitor compound is present in the composition at a concentration of about 0.01% by weight to about 10% by weight.

For example, in certain aspects, the second inhibitor compound is present in the composition at a concentration of about 0.01% by weight, about 0.1% by weight, about 1% by weight, about 5% by weight, about 10% by weight, about 15% by weight, about 20% by weight, about 25% by weight, about 30% by weight, about 35% by weight, about 40% by weight, about 45% by weight, or about 50% by weight.

In some aspects, a mole ratio of the first inhibitor compound to the second inhibitor compound is about 100:1 to about 1:100. In some aspects, a mole ratio of the first inhibitor compound to the second inhibitor compound is about 90:1 to about 1:90. In some aspects, a mole ratio of the first inhibitor compound to the second inhibitor compound is about 80:1 to about 1:80. In some aspects, a mole ratio of the first inhibitor compound to the second inhibitor compound is about 70:1 to about 1:70. In some aspects, a mole ratio of the first inhibitor compound to the second inhibitor compound is about 60:1 to about 1:60. In some aspects, a mole ratio of the first inhibitor compound to the second inhibitor compound is about 50:1 to about 1:50. In some aspects, a mole ratio of the first inhibitor compound to the second inhibitor compound is about 40:1 to about 1:40. In some aspects, a mole ratio of the first inhibitor compound to the second inhibitor compound is about 30:1 to about 1:30. In some aspects, a mole ratio of the first inhibitor compound to the second inhibitor compound is about 20:1 to about 1:20. In some aspects, a mole ratio of the first inhibitor compound to the second inhibitor compound is about 10:1 to about 1:10. In some aspects, a mole ratio of the first inhibitor compound to the second inhibitor compound is about 1:1.

In some aspects, the composition also includes one or more additional compounds selected from the group consisting of 2,2,6,6-tetramethylpiperidin-1-oxyl; 2,2,6,6-tetramethylpiperidin-1-ol; 4-hydroxyl-2,2,6,6-tetramethylpiperidin-1-oxyl; 4-hydroxy-2,2,6,6-tetramethylpiperidin-1-ol; 4-oxo-2,2,6,6-tetramethylpiperidin-1-oxyl; 4-oxo-2,2,6,6-tetramethylpiperidin-1-ol; 4-acetoxy-2,2,6,6-tetramethylpiperidin-1-oxyl; 4-acetoxy-2,2,6,6-tetramethylpiperidin-1-ol; 4-propionoxy-2,2,6,6-tetramethylpiperidin-1-oxyl; 4-propionoxy-2,2,6,6-tetramethylpiperidin-1-ol; and bis((2,2,6,6-tetramethylpiperidin-1-oxyl)-4-yl) oxalate. In some aspects, the composition also includes 2,2,6,6-tetramethylpiperidin-1-oxyl. In some aspects, the composition also includes 2,2,6,6-tetramethylpiperidin-1-ol. In some aspects, the composition also includes 4-hydroxyl-2,2,6,6-tetramethylpiperidin-1-oxyl. In some aspects, the composition also includes 4-hydroxy-2,2,6,6-tetramethylpiperidin-1-ol. In some aspects, the composition also includes 4-oxo-2,2,6,6-tetramethylpiperidin-1-oxyl. In some aspects, the composition also includes 4-oxo-2,2,6,6-tetramethylpiperidin-1-ol. In some aspects, the composition also includes 4-acetoxy-2,2,6,6-tetramethylpiperidin-1-oxyl. In some aspects, the composition also includes 4-acetoxy-2,2,6,6-tetramethylpiperidin-1-ol. In some aspects, the composition also includes 4-propionoxy-2,2,6,6-tetramethylpiperidin-1-oxyl. In some aspects, the composition also includes 4-propionoxy-2,2,6,6-tetramethylpiperidin-1-ol. In some aspects, the composition also includes bis((2,2,6,6-tetramethylpiperidin-1-oxyl)-4-yl) oxalate.

The composition may optionally also include one or more organic solvents. One of ordinary skill in the art will appreciate that there are many organic solvents that are compatible with the compositions of the disclosure. For example, in some aspects, the one or more organic solvents are selected from vinyl acetate, dimethyl phthalate, dimethylformamide, toluene, xylene, highly aromatic naphtha, acetonitrile, ethyl acetate, acetone, dichloromethane, tetrahydrofuran, hexanes, dimethyl sulfoxide, N-methyl-2-pyrrolidone, and combinations thereof. In certain aspects, the composition also includes vinyl acetate. In certain aspects, the composition also includes dimethyl phthalate. In certain aspects, the composition also includes dimethylformamide. In certain aspects, the composition also includes toluene. In certain aspects, the composition also includes xylene. In certain aspects, the composition also includes highly aromatic naphtha. In certain aspects, the composition also includes acetonitrile.

In some aspects, the composition also includes one or more ethylenic unsaturated monomers. One of ordinary skill in the art will appreciate that there are many ethylenic unsaturated monomers that are compatible with the compositions of the disclosure. For example, in some aspects, the one or more ethylenic unsaturated monomers are selected from vinyl acetate, acrylonitrile, acrylates, methacrylates, 1,3-butadiene, styrene, isoprene, (meth)acrylic acid, and combinations thereof. In certain aspects, the composition also includes vinyl acetate. In certain aspects, the composition also includes acrylonitrile. In certain aspects, the composition also includes acrylates. In certain aspects, the composition also includes methacrylates. In certain aspects, the composition also includes 1,3-butadiene. In certain aspects, the composition also includes styrene. In certain aspects, the composition also includes isoprene. In certain aspects, the composition also includes (meth)acrylic acid.

The compositions of the disclosure are stable and remain useful polymerization inhibitors even under acidic conditions. Thus, the compositions of the disclosure are useful for inhibiting the premature polymerization of monomers during manufacturing process, particularly those that are performed under acidic conditions. For example, the compositions of the disclosure are useful for preventing polymerization of acrylates, which may include, but are not limited to, acrylonitrile, acrylic acid, methyl methacrylic acid and its esters, and vinyl acetate.

In some aspects, the composition disclosed herein do not include 4-hydroxy-2,2,6,6-tetramethylpiperidin-1-oxyl (HTEMPO). In some aspects, the composition disclosed herein do not include 4-oxo-2,2,6,6-tetramethylpiperidin-1-oxyl (OTEMPO). In some aspects, the HTEMPO and OTEMPO are not added to ethylenic unsaturated monomers.

### Methods of Using the Compositions of the Disclosure

The present disclosure also relates to methods of inhibiting polymerization of monomers that include adding a composition of the disclosure to the monomer. In some aspects, an effective amount of the composition of the disclosure is added to the monomer, where an effective amount is any amount sufficient to inhibit the polymerization of the monomer.

In some aspects, the monomer is an ethylenic unsaturated monomer. In some aspects the monomer is an ethylenic unsaturated monomer selected from vinyl acetate, acrylonitrile, acrylate esters, methacrylate esters, 1,3-butadiene, styrene, divinyl benzene, isoprene, cyclopentadiene, dicyclopentadiene, acrylic acid, (meth)acrylic acid, and combinations thereof are disclosed. In some aspects, the methods disclosed herein are useful in inhibiting the polymerization of vinyl acetate. In some aspects, the methods disclosed herein are useful in inhibiting the polymerization of acrylonitrile. In some aspects, the methods disclosed herein are useful in inhibiting the polymerization of acrylate esters. In some aspects, the methods disclosed herein are useful in inhibiting the polymerization of methacrylate esters. In some aspects, the methods disclosed herein are useful in inhibiting the polymerization of 1,3-butadiene. In some aspects, the methods disclosed herein are useful in inhibiting the polymerization of styrene. In some aspects, the methods disclosed herein are useful in inhibiting the polymerization of isoprene. In some aspects, the methods disclosed herein are useful in inhibiting the polymerization of acrylic acid. In some aspects, the methods disclosed herein are useful in inhibiting the polymerization of (meth)acrylic acid.

The composition of the disclosure can be added manually or automatically to the fluid. The composition can also be added continuously and/or intermittently. Automatic addition may be accomplished through the use of chemical injection pumps. The chemical injection pumps may be programmed to add particular amounts of the polymerization inhibitor composition, or any components thereof, at certain time intervals to the fluid. In alternate aspects, the chemical injection pumps can be manually controlled to add particular amounts of the polymerization inhibitor composition, or any components thereof, to the fluid. Addition of the presently disclosed polymerization inhibitor compositions to the monomer will thereby inhibit polymerization of the monomer.

In some aspects, the monomer is provided as a neat liquid. In other aspects, the monomer is provided within a solution, hereafter referred to as “the monomer solution.”

In some aspects, the monomer solution also includes one or more additional components selected from an acid, an organic solvent, water, and combinations thereof. For example, in some aspects, the monomer solution includes one or more organic solvents selected from vinyl acetate, dimethyl phthalate, dimethylformamide, toluene, ethyltoluene, xylene, highly aromatic naphtha, acetonitrile, ethyl acetate, acetone, dichloromethane, tetrahydrofuran, hexanes, dimethyl sulfoxide, N-methyl-2-pyrrolidone, and combinations thereof. In some aspects, the monomer solution includes one or more acids selected from hydrochloric acid, hydrofluoric acid, hydrobromic acid, hydroiodic acid, nitric acid, phosphoric acid, sulfuric acid, boric acid, perchloric acid, formic acid, acetic acid, propionic acid, butyric acid, valeric acid, caproic acid, ethanic acid, caprylic acid, undecylic acid, lauric acid, oxalic acid, malonic acid, succinic acid, glutaric acid, adipic acid, pimelic acid, and suberic acid. In some aspects, the monomer solution includes water.

In some aspects, the monomer solution has a pH value of about 1 to about 7. In some aspects, the monomer solution has a pH value of about 1 to about 6. In some aspects, the monomer solution has a pH value of about 2 to about 6. In some aspects, the monomer solution has a pH value of about 3 to about 6. In some aspects, the monomer solution has a pH value of about 4 to about 6. In some aspects, the monomer solution has a pH value of about 5 to about 6.

In some aspects, the composition is added to the monomer such that a concentration of the first inhibitor compound is about 0.1 ppm to about 10,000 ppm. In some aspects, the composition is added to the monomer such that a concentration of the first inhibitor compound is about 0.1 ppm to about 5,000 ppm. In some aspects, the composition is added to the monomer such that a concentration of the first inhibitor compound is about 0.1 ppm to about 1,000 ppm. In some aspects, the composition is added to the monomer such that a concentration of the first inhibitor compound is about 0.1 ppm to about 500 ppm.

In some aspects, the composition is added to the monomer such that a concentration of the second inhibitor compound is about 0.1 ppm to about 10,000 ppm. In some aspects, the composition is added to the monomer such that a concentration of the second inhibitor compound is about 0.1 ppm to about 5,000 ppm. In some aspects, the composition is added to the monomer such that a concentration of the second inhibitor compound is about 0.1 ppm to about 1,000 ppm. In some aspects, the composition is added to the monomer such that a concentration of the second inhibitor compound is about 0.1 ppm to about 500 ppm.

The methods of the disclosure are useful for inhibiting the premature polymerization of monomers during manufacturing process, particularly those that are performed under acidic conditions. For example, the methods of the disclosure are useful for preventing polymerization of acrylates, which may include, but are not limited to, acrylonitrile, acrylic acid, methyl methacrylic acid and its esters, and vinyl acetate.

The methods of the disclosure are also useful for preventing the premature polymerization of styrene during manufacturing and purification processes.

The methods of the disclosure are also useful in butadiene extraction processes. This utility stems from the balanced partition coefficients between polar organic phases and organic phases.

In some aspects, the compositions disclosed herein are used in distillative purification of olefins. For example, the composition can be added to the process stream before entering the distillation unit or the composition can be added to the process stream in the distillation unit.

### Processes for Preparing Polymerization Inhibitors of the Disclosure

The present disclosure also relates to processes for preparing a compound of formula (III):

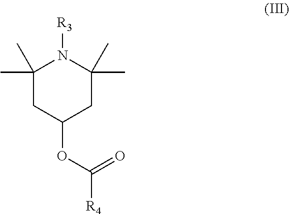

wherein:
R₃ is —O· or —OH; and
R₄ is C₁-C₂₂ alkyl, C₁-C₂₂ alkenyl, C₁-C₂₂ alkynyl, C₁-C₂₂ cycloalkyl, aryl, and C₁-C₂₂ alkylene aryl, wherein the cycloalkyl and aryl are optionally substituted with one or more C₁-C₂₂ alkyl, C₁-C₂₂ alkenyl, C₁-C₂₂ alkynyl, or aryl.

In some aspects, the process for preparing a compound of formula (III) includes treating a compound of formula (IIIa):

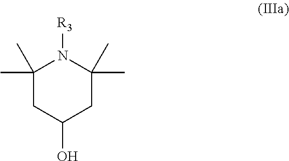

with a compound of (IIIb):

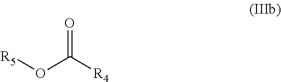

wherein R₅ is C₁-C₂₂ alkyl or C₁-C₂₂ alkenyl,
   within a solution, to afford the compound of formula (III).

In some aspects, R₃ is —O·. In some aspects, R₃ is —OH.

In some aspects, R₄ is C₁-C₂₂ alkyl. In some aspects, R₄ is C₁-C₁₂ alkyl. In some aspects, R₄ is C₁-C₆ alkyl. In some aspects, R₄ is ethyl. In some aspects, R₄ is methyl.

In one aspect, R₃ is —O· and R₄ is methyl. In another aspect, R₃ is —O· and R₄ is ethyl. In another aspect, R₃ is —OH and R₄ is methyl. In another aspect, R₃ is —OH and R₄ is ethyl.

In some aspects, R₅ is C₁-C₂₂ alkyl. In some aspects, R₅ is C₁-C₁₂ alkyl. In some aspects, R₅ is C₁-C₆ alkyl.

In some aspects R₅ is C₁-C₂₂ alkenyl. In some aspects, R₅ is C₁-C₁₂ alkenyl. In some aspects, R₅ is C₁-C₆ alkenyl. In some aspects, R₅ is C₂ alkenyl.

In some aspects, the process for preparing a compound of formula (III) includes treating a compound of formula (IIIa):

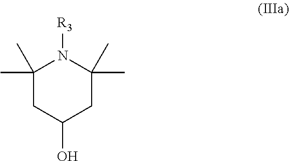

with a compound of (IIIc):

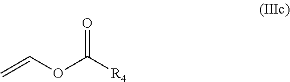

within a solution, to afford the compound of formula (III).

In some aspects, the compound of formula (IIIa) is treated with the compound of formula (IIIb) in the presence of a catalyst and heat. In some aspects, the compound of formula (IIIa) is treated with the compound of formula (IIIc) in the presence of a catalyst and heat. One of ordinary skill will appreciate that there are many appropriate catalysts that can be used to form a compound of formula (III) by treating a compound of formula (IIIa) with a compound of formula (IIIb) or (IIIc). For example, in some aspects, the catalyst is an amine-containing compound. In certain aspects, the catalyst is 4-dimethylaminopyridine, also known as DMAP. In some aspects, the solution of the compound of formula (IIIa) and (IIIb), or (IIIc), is heated to a temperature of about 50° C. to about 100° C. In some aspects, the solution is heated to a temperature of about 50° C. to about 85° C.

In some aspects, the process for preparing a compound of formula (III) also includes purging the solution with a stream of nitrogen. In some aspects, the step of purging the solution with a stream of nitrogen is performed concurrently with the step of treating a compound of formula (IIIa) with a compound of formula (IIIb) to afford the compound of formula (III). In some aspects, the step of purging the solution with a stream of nitrogen is performed concurrently with the step of treating a compound of formula (IIIa) with a compound of formula (IIIc) to afford the compound of formula (III). In some aspects, the step of purging the solution with a stream of nitrogen is performed after the step of treating a compound of formula (IIIa) with a compound of formula (IIIb) to afford the compound of formula (III). In some aspects, the step of purging the solution with a stream of nitrogen is performed after the step of treating a compound of formula (IIIa) with a compound of formula (IIIc) to afford the compound of formula (III). Without being bound by theory, the step of purging the solution with a stream of nitrogen may be useful in removing certain reaction byproducts that pushes the reaction equilibrium towards formation of the compound of formula (III).

### EXAMPLES

### Example 1: High Severity Static Performance Blank

Commercial styrene is shipped and stored after treatment with 4-tert-butylcatechol (TBC) to prevent gum formation. Thus, prior to using the commercial styrene in the performance test, TBC was removed by elution through an alumina column thereby yielding fresh styrene. The styrene was diluted with toluene to a solution comprising about 70% (w/w) styrene and about 30% (w/w) toluene. Into 24 pressure-resistant glass tubes, about 10 mL of the styrene solution were added per tube. After the removal of dissolved oxygen in the solutions by bubbling through the solutions nitrogen gas for about 2 minutes, PTFE screw caps armed with fluoroelastomer (FETFE) O-rings were used to cap the test tubes. All the tubes were placed into a heating block preheated to about 135° C. After the first 15 minutes had elapsed, four reactor tubes were pulled from the heating block and, thereafter, at time intervals of 15 minutes for a total reaction time of about 90 minutes. To quench the polymerization, the four tubes were immediately placed in an ice-bath followed by the immediate dilution of the reaction mixture with toluene. A proprietary method was used to determine the concentration of the soluble polymer product in the reaction mixture.

### Example 2: High Severity Static Performance 25 ppm of HTEMPO

A freshly prepared solution of commercial styrene and toluene was treated with 25 ppm of HTEMPO. The reaction kinetics of this solution was performed as stated in Example 1.

### Example 3: High Severity Static Performance 25 ppm of 4-Acetoxy TEMPO

A 25-ppm dose of 4-Acetoxy TEMPO was added into the toluene-diluted styrene solution prepared in accordance with the procedure in Example 2. Likewise, the performance test and polymer analysis were carried out as in Example 1.

### Example 4: High Severity Static Performance 12.5 ppm of HTEMPO and 12.5 ppm PDA

After treating the toluene-styrene solution with 12.5 ppm of HTEMPO and 12.5 ppm of the PDA prototype N,N′-di-sec-butyl phenylenediamine, the antipolymerant performance test was carried out as outline in the above examples.

### Example 5: High Severity Static Performance 12.5 ppm of 4-Acetoxy TEMPO and 12.5 ppm PDA

As in Example 4, a reaction solution mixture of styrene and toluene was dosed with 12.5 ppm of 4-Acetoxy TEMPO and 12.5 ppm of the prototype PDA. The kinetics study was conducted much like in the foregoing examples.

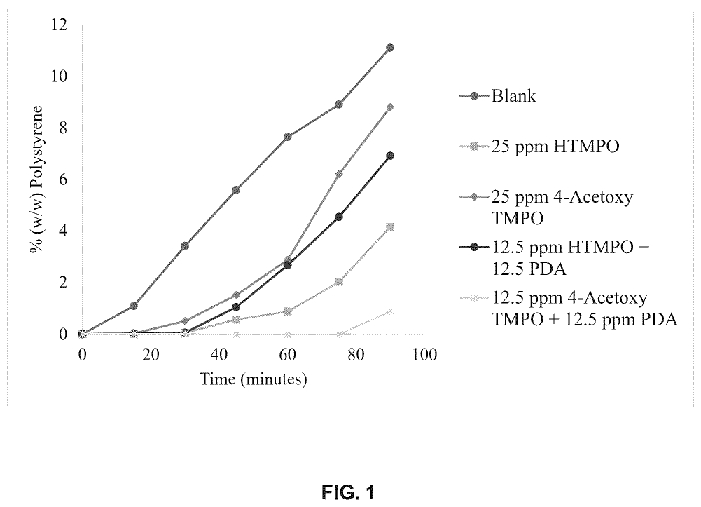

The results of the comparative study are shown in FIG. 1. According to the test, it is very evident that the normally effective free radical polymerization inhibitors are less effective at temperatures above the industry's operational conditions. HTEMPO has a short induction time when the polymerization takes place at 135° C. Similarly, as is shown in the kinetics trend in FIG. 1, 4-Acetoxy TEMPO exhibits comparatively less efficiency as an inhibitor.

The combined used of 12.5 ppm of HTEMPO and 12.5 ppm of the PDA antipolymerant did not yield antipolymerant performance better than HTEMPO. By contrast, dosing the reaction solution with 12.5 ppm of 4-Acetoxy TEMPO and 12.5 ppm of the PDA resulted in a very significant improvement in preventing polymerization at the uncharacteristically high temperature of 135° C.

### Example 6: CSTR Performance Test Using Incumbent Product

During the processing and purification of reactive monomers, a process stream is continuously fed into the process column and, likewise, the same stream transferred out of the processing equipment. Thus, the operations are under a continuous flow of the process stream. To simulate the online process in the reboiler section, or bottom, of the tower of the process tower, a continuously stirred tank reactor (CSTR) was used in lieu of capped tubes in the above examples.

As previously stated in example 1, commercial styrene was freshly prepared by removing t-butyl catechol using an alumina column. Instead of using neat styrene, it had to be diluted with a suitable solvent that has a boiling point above 150° C. Since it boils at about 162° C., p-ethyltoluene (pET), was used as a diluent. The dilution of the styrene was to slow down the rate of polymerization at 135° C., the high severity temperature in the procedure. A portion of this styrene, weighing 1400 grams was diluted with p-ethyl toluene. To the 1400 grams of the freshly prepared styrene, 600 grams of p-ethyltoluene were added to yield the untreated hydrocarbon stream. The solution thus comprised about 70% (w/w) styrene and about 30% (w/w) p-ethyl toluene. N,N′-di-sec-butyl phenylenediamine, about 0.0100 grams, was added to the solution followed by about 0.0100 grams of 2,6-di-t-butylphenol. This constituted the treatment of the reaction solution with about 100 ppm of the incumbent antipolymerant.

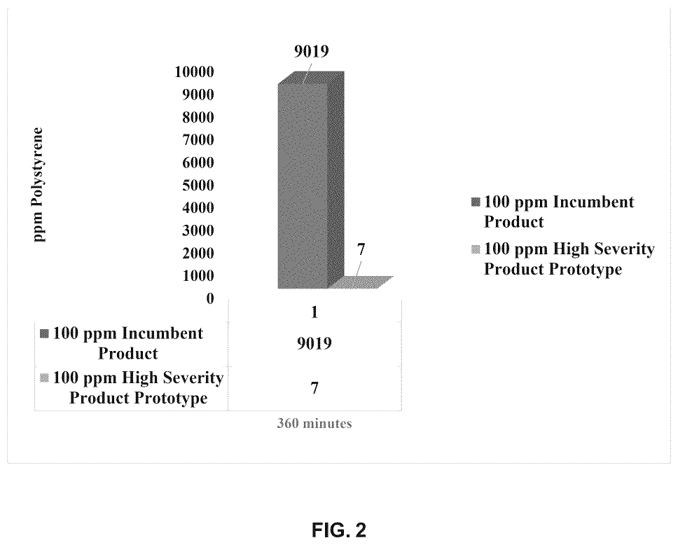

Of the treated solution, about 65 mL were added to a continuously stirred tank reactor (CSTR). The CSTR was fitted with an inlet transfer line through which a pump added the reaction mixture into the reactor. An outlet transfer line was also attached to the outlet port of the CSTR. To this line was attached a second pump that carried the effluent stream from the reactor. Both pumps were adjusted to result in the CSTR residence time of about 15 minutes. The reactor was placed on an isomantle heating block, and the reaction temperature set at 135° C. using a thermocouple probe inserted directly into the reaction mixture. Since the addition of p-ethyltoluene imparted a high boiling point to the reaction solution, no evaporation or loss of reaction solution was observed. Nonetheless, a reflux setup was attached to the reactor to prevent losses of the reaction solution due to evaporation. As soon as the reaction temperature reached about 135° C., a sample of the effluent stream was collected. From that point onwards, a sample was collected at a time interval of about 30 minutes. Using a proprietary method, the polymer content in the effluent stream was analyzed. After a total CSTR reaction time of about 6 hours, the average concentration of polystyrene was about 9019 ppm (see FIG. 2).

### Example 7: CSTR Performance Test Using New High-Severity Product

The same procedure used in Example 6 was used to prepare and test the performance of 100 ppm of the high severity product prototype. The product comprised about 50% (w/w) 4-acetoxy TEMPO and about 50% (w/w) N,N′-di-sec-butyl phenylenediamine. The concentration of polystyrene after about 6 hours was about 7 ppm. This was within the background polymer level of styrene under ambient conditions.

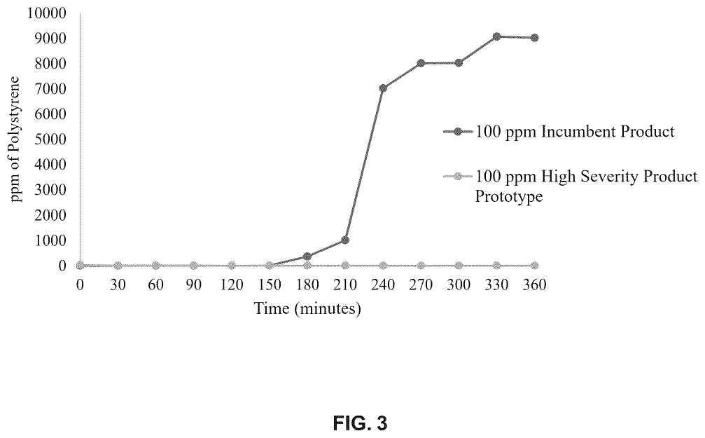

The bar graph of the results is shown in FIG. 2, and the polymerization kinetics trend is shown in FIG. 3. From these results, it is quite clear that the prototype of the new antifoulant composition exhibits a significantly better antipolymerant efficacy than the incumbent (50% (w/w) PDA and 50% (w/w) hindered phenol). The high-severity antipolymerant effectively inhibits polymerization at high-severity temperature.

All of the compositions and methods disclosed and claimed herein can be made and executed without undue experimentation in light of the present disclosure. While this invention may be embodied in many different forms, there are described in detail herein specific preferred aspects of the invention. The present disclosure is an exemplification of the principles of the invention and is not intended to limit the invention to the particular aspects illustrated. In addition, unless expressly stated to the contrary, use of the term “a” is intended to include “at least one” or “one or more.” For example, “a compound” is intended to include “at least one compound” or “one or more compounds.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

As used herein, the term “about” refers to the cited value being within the errors arising from the standard deviation found in their respective testing measurements, and if those errors cannot be determined, then “about” may refer to, for example, within 5% of the cited value.

Furthermore, the invention encompasses any and all possible combinations of some or all of the various aspects described herein. It should also be understood that various changes and modifications to the presently preferred aspects described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A composition for inhibiting monomer polymerization, comprising:
a first inhibitor compound comprising 4-acetoxy TEMPO; and
a second inhibitor compound comprising N,N′-di-sec-butyl phenylenediamine.

2. The composition of claim 1, wherein the first inhibitor compound is present in the composition at a concentration of about 0.01% by weight to about 80% by weight.

3. The composition of claim 1, wherein the second inhibitor compound is present in the composition at a concentration of about 0.01% by weight to about 50% by weight.

4. The composition of claim 1, wherein a mole ratio of the first inhibitor compound to the second inhibitor compound is about 100:1 to about 1:100.

5. The composition of claim 1, wherein the composition further comprises an organic solvent.

6. The composition of claim 1, wherein the composition further comprises an ethylenic unsaturated monomer selected from the group consisting of vinyl acetate, acrylonitrile, an acrylate, a methacrylate, 1,3-butadiene, styrene, isoprene, cyclopentadiene, dicyclopentadiene, acrylic acid, methacrylic acid and any combination thereof.

7. A method of inhibiting polymerization of a monomer, comprising:
adding the composition of claim 1 to the monomer.

8. The method of claim 7, wherein the monomer is provided within a solution.

9. The method of claim 8, wherein the solution further comprises an additional component selected from an acid, an organic solvent, and/or water.

10. The method of claim 7, wherein the monomer is an ethylenic unsaturated monomer.

11. A composition for inhibiting monomer polymerization, comprising:
a first inhibitor compound comprising sebacate dinitroxide; and
a second inhibitor compound comprising N-sec-butyl-N′-phenylphenylenediamine.

12. The composition of claim 11, wherein the first inhibitor compound is present in the composition at a concentration of about 0.01% by weight to about 80% by weight and/or wherein the second inhibitor compound is present in the composition at a concentration of about 0.01% by weight to about 50% by weight.

13. The composition of claim 11, wherein a mole ratio of the first inhibitor compound to the second inhibitor compound is about 100:1 to about 1:100.

14. The composition of claim 11, wherein the composition further comprises an organic solvent.

15. The composition of claim 11, wherein the composition further comprises an ethylenic unsaturated monomer selected from the group consisting of vinyl acetate, acrylonitrile, an acrylate, a methacrylate, 1,3-butadiene, styrene, isoprene, cyclopentadiene, dicyclopentadiene, acrylic acid, methacrylic acid and any combination thereof.

16. A method of inhibiting polymerization of a monomer, comprising:
adding the composition of claim 11 to the monomer.

17. The method of claim 16, wherein the monomer is provided within a solution.

18. The method of claim 16, wherein the solution further comprises an additional component selected from an acid, an organic solvent, and/or water.

19. The method of claim 16, wherein the monomer is an ethylenic unsaturated monomer.
